(19) United States
(12) Patent Application Publication
(10) Pub. No.: US 2008/0243154 A1
(43) Pub. Date: Oct. 2, 2008

(54) Surgical Instrument of the Ablation of Diseased Veins, Particularly Saphenous Veins

(75) Inventor: Maxime Formichi, Marseille (FR)

Correspondence Address: EGBERT LAW OFFICES, 412 MAIN STREET, 7TH FLOOR, HOUSTON, TX, 77002, US

(73) Assignee: VASCULAR OFFICE (SARL), Marseille Cedex 13 (FR)

(21) Appl. No.: 12/063,027

(22) PCT Filed: Aug. 10, 2005
(86) PCT No.: PCT/FR05/02068

§ 371 Date: Mar. 11, 2008

(51) Int. Cl. A61B 17/22

(52) U.S. Cl. 606/159

(57) ABSTRACT

The invention relates to a surgical instrument for the ablation of diseased veins, particularly saphenous veins. The surgical instrument includes a stripping member or olive-shaped member, a traction probe, and a return thread, which can be used to move the olive in opposite directions. In addition, the anterior part of the olive, according to the direction of movement of same during the application of the operating method, is equipped with an axial cavity which is open at the front end of the anterior part, so as to define a circular stripping lip. The stripping olive includes an essentially-cylindrical anterior part and a posterior part having a circular cross-section, which are defined by a circular recess. The circular front end of the posterior part has a diameter greater than that of the anterior part.

Surgical instrument for the ablation of diseased veins, particularly saphenous veins.

This invention concerns a surgical instrument for the ablation of diseased veins and more specifically a surgical device for application of the method for removal saphenous veins (saphenectomy) through pulling, a procedure also called stripping.

Various operating methods based on the stripping technique are well-known and practiced at this time and more specifically, the invention can advantageously be applied to the implementation of the round-trip retrograde stripping method described in a prior French patent of the Inventor, N⁰ 89/09358 (FR-2.649.309).

The surgeon has a surgical instrument called stripper available to perform the ablation of saphenous veins according to the above-mentioned method. Under FR-2.649.309 document, this stripping instrument or stripper consists of a pulling component called an “olive” in the professional language and of two flexible probes equipped at each end with an olive-removable end fitting, these two probes (traction probe and return probe) having a length greater than the length of the veins that might be removed through pulling.

The stripping method practiced with such stripper is described below, using as an example the total ablation of the long saphenous vein, the most common procedure.

The surgeon makes an approx. 2mm first incision at the ankle and a second approx. 2-3 cm incision at the groin crease line. At the ankle, the long saphenous vein is isolated, the lumen of the vein is opened, the surgeon inserts the traction probe provided with a fitting at each end that is led to the groin crease inside the vein, a junction area of the long saphenous vein into the deep rete. The saphenous vein is cut at that level and the traction probe is taken out of the open lumen. The pulling olive is then connected, on one hand to the traction probe coming out at the groin crease line, and on the other hand, to the return probe. One pulls on the other end of the traction probe located at the ankle so that the olive moves toward the ankle, which causes the vein to become detached from the neighboring adherent tissues. The movement of the olive is stopped before it reaches the incision made at the ankle, i.e., approx. 5 cm from the latter so as to limit any injury to the saphenous nerve due to the running of the olive. The saphenous vein is then cut at that level and depending on the practitioner, it is:
either connected through a thread to the traction probe and the saphenous vein
traction probe—olive assembly is led back through and out of the groin crease line by pulling on the return probe attached beforehand to the olive.
or extracted by itself through the incision made at the ankle, the traction probe and the olive being led back and out through the incision made at the groin crease line.

Under this technique, it is important to insert and take out the olive—the bulkiest component of the device—through the incision made in the groin crease line as the resulting scar will be hidden, contrary to the incisions made at the ankle that are highly apparent and visible. Limited to 2 mm, the incision made at the ankle in the end is thus used only to run the traction probe and its end fitting.

Furthermore, if one takes special precautions to stop the movement of the olive and therefore the pulling of the vein a few centimeters from the incision made at the ankle, one avoids injuring the saphenous nerve, which is responsible for postoperative handicapping neuralgia.

U.S. Pat. No. 3,185,155 document describes a stripping device comprising a pulling head and a single probe equipped with end fittings. The anterior end of the pulling head is provided with a cavity delimiting a pulling lip and in which the vein can collect when pulled. However, the very small volume of this cavity does not guarantee that it can be used as collecting reservoir for a significant length of the vein being pulled so that it is possible that, after a few centimeters of the vein are pulled, the pulling lip no longer fulfills its function, resulting in an undesirable invagination (a stripping technique using a different concept under which the vein is deliberately turned inside out like a glove finger). Furthermore, the device described in this document is a one-directional stripper that does not offer any possibility of bringing the pulling head back to its point of insertion.

In practice, the use of a stripper and the implementation of a conventional stripping method (for example U.S. Pat. No. 3,185,155) require to make an incision of approx. 15-20 mm at the ankle, causing at a later time the formation of a highly visible unattractive scar.

The purpose of the invention is a stripping surgical instrument or stripper to implement the round-trip retrograde stripping method and featuring improvements making it possible to:
make at the ankle an incision of a size approx. ten times less large than that of the incision required by the use of conventional strippers and methods;
reduce injury to the neighboring tissues during the vein pulling action;
simplify manipulations during the operating procedure.

Under the invention, these goals are achieved using a stripping device of a type comprising a pulling component or pulling olive, a traction probe and a return thread to move said olive in the opposite direction and under which the anterior part of the olive, considering the active movement direction of the latter during application of the operating method, is provided with an axial cavity open at the front end of said anterior part, this device being noteworthy in that the pulling olive comprises a basically cylindrical anterior part and a posterior part delimited by a circular groove, the circular front end of said posterior part having a diameter greater than the diameter of said anterior part.

Using this arrangement, the circular groove and the slightly larger diameter of the circular front end of the olive posterior part in relation to the diameter of anterior part of the latter constitute additional locking means efficiently opposing invagination.

According to another characteristic arrangement, the removal olive designed to permit the return of the olive toward the inlet incision is permanently integral with the posterior part of the olive.

Manipulations that have been necessary to date to couple or uncouple the olive and the probe are thus eliminated.

The above purposes, characteristics and advantages and many more will become clearer from the description below and the attached drawings where:

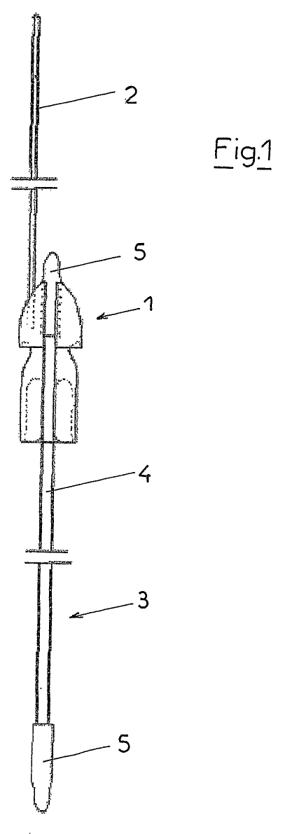

FIG. 1 is an elevation view of a first embodiment of a stripping surgical instrument or stripper under the invention, in which the two component parts are shown in coupling state.

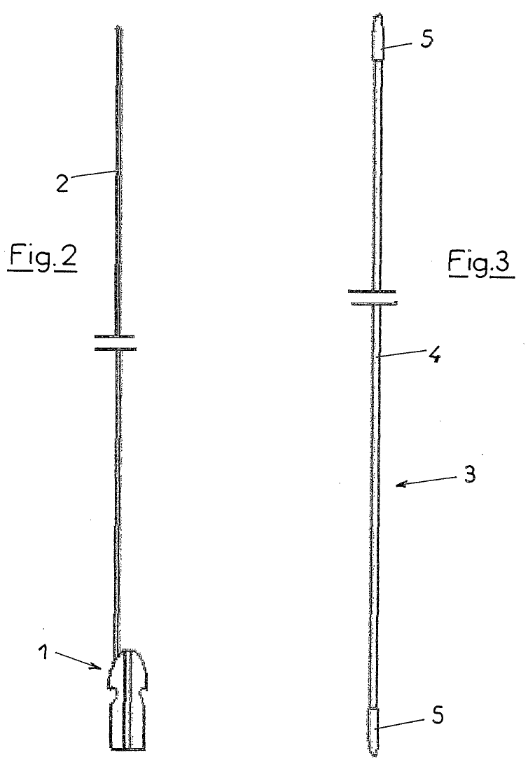

FIGS. 2 and 3 show, separately and respectively, the pulling olive and its return probe or thread and the traction probe.

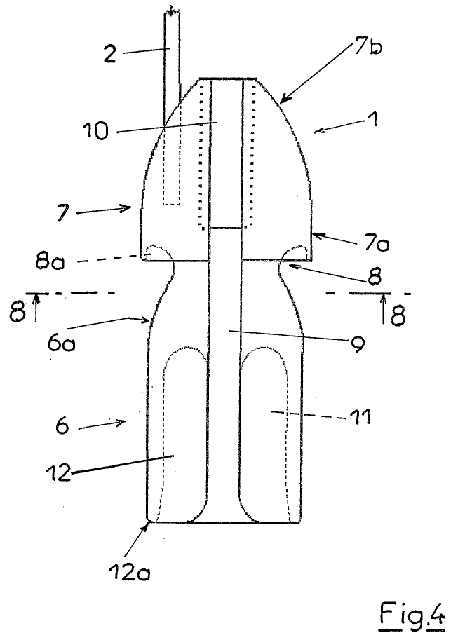

FIG. 4 is a front view of the pulling olive.

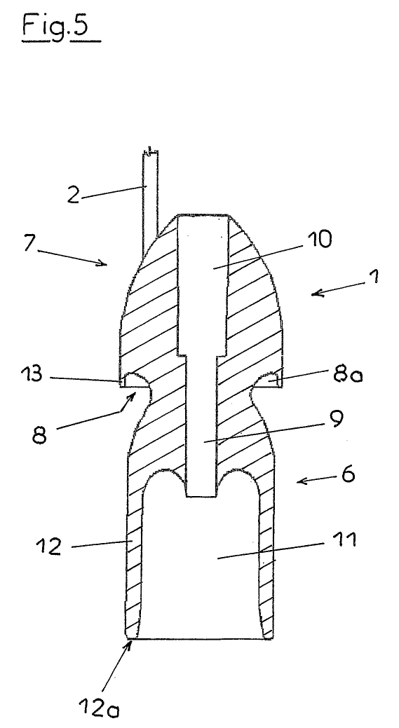

FIG. 5 is an axial cross-section of said olive.

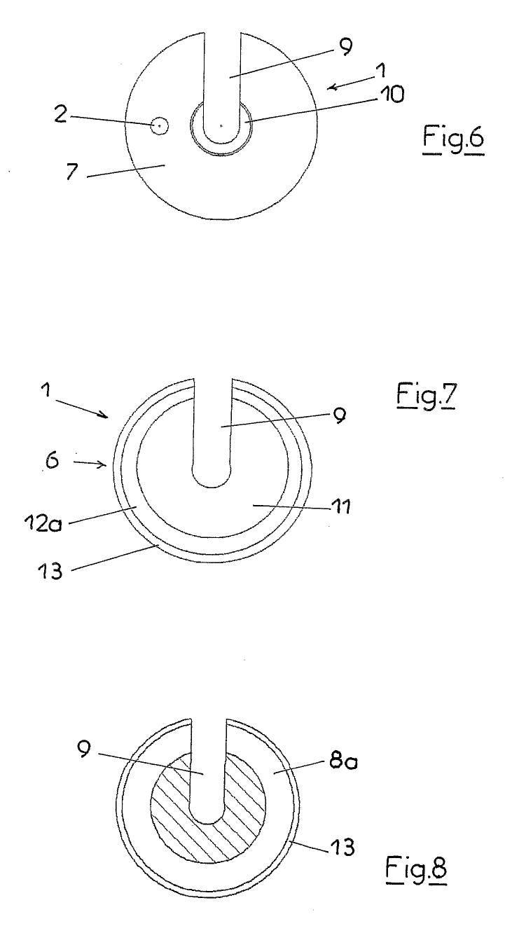

FIG. 6 is a top view of FIG. 4.

FIG. 7 is a bottom view of FIG. 4.

FIG. 8 is a sectional view along line 8-8 from FIG. 4.

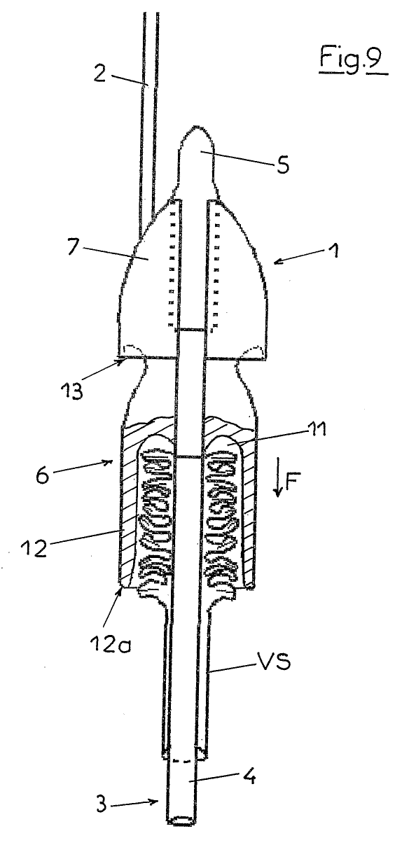

FIG. 9 is an elevation view with partial axial cross-section and at a larger scale, showing the vein collection (for example of the crural portion of the saphenous vein), in the cavity or compartment of the pulling olive.

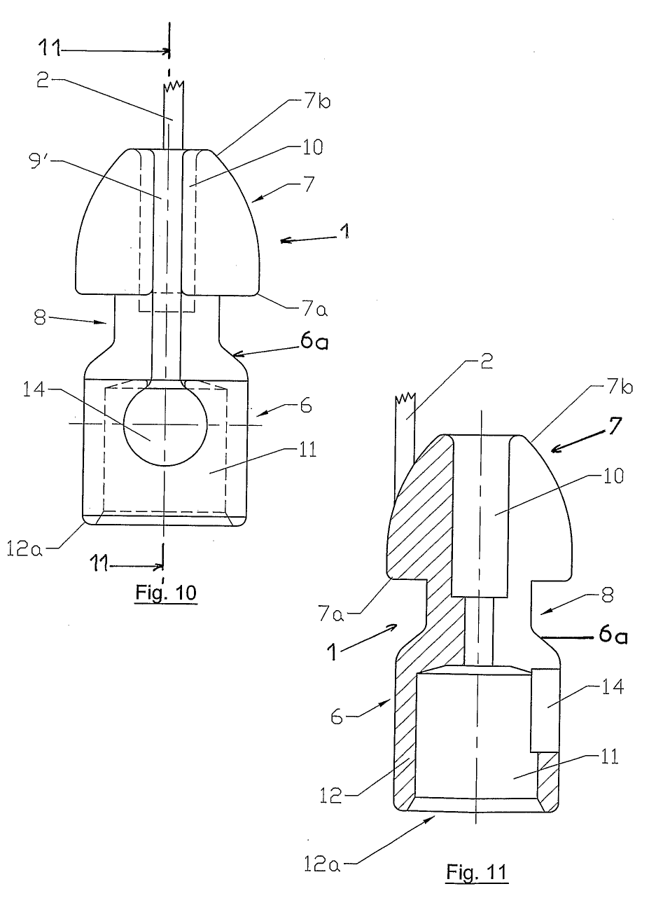

FIG. 10 is a front view of another interesting embodiment of the pulling olive under the invention and of its return probe or thread partially shown.

FIG. 11 is an axial sectional view along line 11-1 1 from FIG. 10.

FIG. 12 is a top view of FIG. 11.

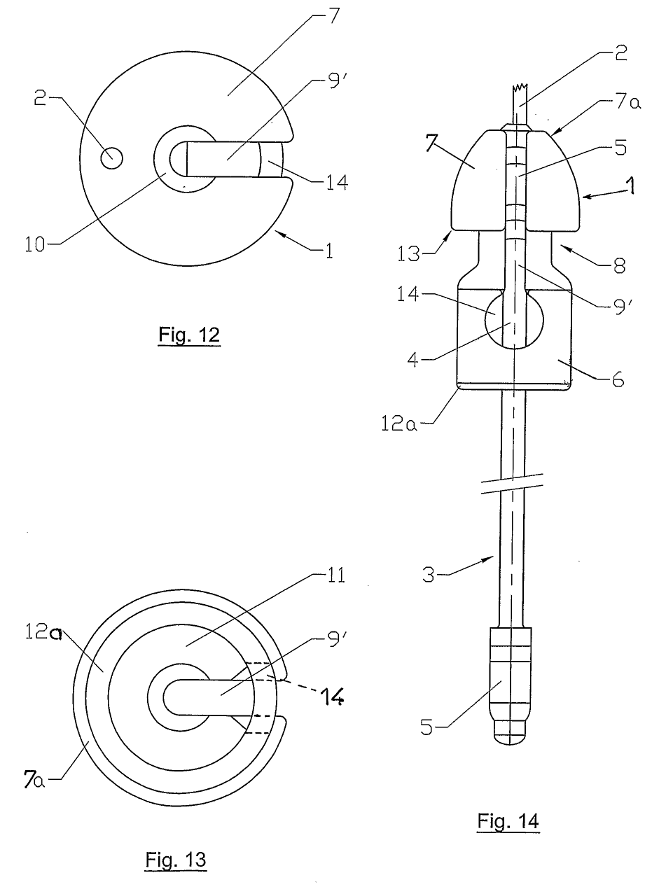

FIG. 13 is a bottom view of FIG. 11.

FIG. 14 is an elevation view of this second embodiment of the stripping surgical instrument in which both component parts are shown in coupling state.

Reference is made to those drawings to describe two interesting, but in no way limiting, embodiments of the stripping surgical instrument or stripper under the invention.

On those drawings, identical components of both embodiments are given the same number references.

In the outline below, terms such as “anterior”, “front”, “posterior”, “rear”, etc., considering the active movement direction of the olive during the pulling phase aiming at detaching the vein from the surrounding adherent tissues are used to describe the pulling olive.

Furthermore, to describe the invention, reference is made to an application thereof to the ablation of the long or great saphenous vein; however, it is obvious that the described instrument can be used for the ablation of other similar vessels.

The instrument under the invention consists of a pulling olive 1 permanently integral with a return probe or thread 2 and of a traction probe 3, capable of being connected in a removable manner to said olive.

The traction probe 3 consists of a flexible thread 4 equipped at each end with a fitting 5 designed to:
facilitate venous catheterization;

achieve anchoring means to attach the thread to the olive that is the component to carry out the ablation of the vein when one pulls on the probe.

The thread 4 and end fittings 5 can be made out of any suitable materials, e.g., polyamide. The thread 4 can be approx. 1000 mm long and have a diameter of approx. 2 mm. The end fittings can be 15 mm long and have a diameter of approx. 3.4 mm over the major part of their length; they have a tapered free end in the shape of an ogive, with a diameter of approx. 2.6 mm to promote catheterization of the vein, the larger body of said fittings being used for removable anchoring of the traction probe 3 onto the olive 1.

The probe 3 must be able to withstand a traction of 30-50 Newton; the thread 4 must not break or be deformed when working under traction.

The probe 3 can be made of one single piece (thread 4+ end fittings 5) using any suitable over-moulding technique. Alternatively, the junction of the end fittings and of the thread can be achieved through gluing or sealing, for example, ultrasound sealing.

The probe 3 is coupled in a removable manner to the olive 1, through one of its end fittings 5.

The olive has an oblong shape, comprising a basically cylindrical anterior portion 6 and a posterior portion with circular section 7, preferably of a streamlined form to allow for the removal of the device and of the vein without any possibility of getting hooked to the surrounding tissue when moving. It is made out of a hard deformation-resistant material, compatible with being integral with the removal thread that can be made out of polyamide. The olive 1 is for example made out of acetal homopolymer (marketed under the registered trademark “Delrin”) or any other equivalent material.

The olive can be approx. 30 mm long. According to a characteristic arrangement of the invention, it features in its middle, or roughly middle, part a circular groove 8 delimiting its basically cylindrical anterior part 6 and its posterior part of preferably cylindro-ogival shape 7 comprising a cylindrical front portion 7a and an ogival rear portion 7b. The circular front end 7a of the posterior part 7 also has a diameter greater than the diameter of the anterior part 6.

For example, the anterior part 6 can have a 10 mm diameter while the front end 7a of the posterior part 7, for example consisting of the cylindrical portion thereof, can have a diameter slightly larger than that of said anterior part, for example 11 mm.

The rear portion 6a connecting the anterior part 6 of the olive 1 to the posterior part 7 thereof has a tapered shape, for example a roughly ogival shape.

The return thread 2 is solidly and permanently attached, through one of its ends and using any suitable method, onto the posterior end of the olive. The junction between the removal thread 2 and the olive 1 must be capable of withstanding a tensile force of 25 Newton. The removal thread can be approx. 1000 mm long and have a diameter of approx. 1 mm. It can be made out of a material identical to that of the thread 4 of the traction probe 3, for example polyamide, or any other biocompatible suitable material.

According to the embodiment shown in FIGS. 1 through 9, the olive 1 comprises laterally a longitudinal slot 9 extending over its whole length and whose width is designed so that the thread 4 of the traction probe 3 can be run through it when the probe is hooked to the olive. This slot 9 communicates with an axial compartment 10 open at its ends, made in the posterior part of the olive and shaped to receive and hold one of the end fittings 5 of the traction probe when coupling the two components of the stripper. The length of this axial compartment 10 corresponds roughly to the length of the cylindrical portion of said end fittings and its diameter permits to house, without excessive gap, one of said end fittings. The olive 1 must not become deformed; it must block the traction probe and withstand traction forces, for example traction forces from 30 to 50 Newton.

In the cylindrical anterior part 6 of the olive 1, a cylindrical cavity 11 is provided, that is open at the front end of said olive, this cavity being delimited by a cylindrical skirt 12 and its depth is preferably barely smaller than, or approximately equal to the length of said part. The depth of the axial cavity 11 is at least equal to one fifth of the total length of the olive 1, or at least equal to one third of the length of the anterior portion 6 of said olive. The depth of this cavity must be at least sufficient to accommodate the first vein segment pulled.

The free front edge 12a of the cylindrical skirt 12 constitutes a circular pulling lip while the cylindrical cavity 11 is used collect the vein upon the active movement of the olive pulled by the traction probe 3 so as to prevent invagination of the olive inside the vessel during ablation.

In case of any starting invagination, the circular groove 8 and the slightly greater diameter of the circular end 7a of the posterior part 7 of the olive in relation to the diameter of the anterior part 6 of the latter constitute additional locking means opposing invagination.

The circular groove can advantageously be shaped so as to promote this locking action.

For example, the olive 1 can comprise a second circular lip 13 located behind and away from the cylindrical anterior part 6. This circular lip 13 oriented toward the front consists of an extension or projection of the cylindrical portion 7a of the posterior part 7 of the olive. Therefore, it has a diameter slightly greater than that of the anterior part 6 of the olive and extends forward in the direction of the circular groove 8, so that a part of it delimited by said lip goes into the posterior part 7 of the olive, thus constituting an annular pocket 8a located in the rear part of the groove 8.

The olive 1 thus comprises two successive pulling lips 12a, 13, respectively, the posterior lip 13 catching up the vein when it goes past the anterior part 6 of said olive, in a manner comparable to a two-blade razor.

To use the ablation instrument or stripper under the invention, the surgeon proceeds in the manner indicated in the introduction part of this description.

Considering the advantageous application of the invention to the ablation of a diseased saphenous vein designated with the letter reference “VS” in FIG. 9, the surgeon makes an approx. 2-3 mm first incision at the ankle and an approx. 2-3 cm incision at the groin crease line. At the ankle, the saphenous vein is isolated, the lumen of the vein is opened, the surgeon inserts the traction probe 3 through one of its ends provided with a fitting 5, that is led to the groin crease line, inside the vein—an area junction of the saphenous vein into the deep rete. The saphenous vein is cut at that level and the probe 3 is taken out of the open lumen. The pulling olive 1 is then connected to the fitting 5 of the traction probe 3 end coming out at the groin creasing line. One pulls on the other end of the probe 3 that is located at the ankle so that the olive moves toward the ankle (per the arrow F on FIG. 9), which causes the vein to become detached from the neighboring adherent tissues and to collect the length of detached vein in the axial cavity 11 of the olive. When moving, the latter draws at the same time the return thread 2 integral with its posterior part 7. The movement of the olive 1 is stopped before it reaches the incision made at the ankle, i.e., approx. 5 cm from said incision. The surgeon then pulls on the return thread 2 accessible at the groin so as to move in the opposite direction the olive that draws the vein VS detached from the adherent tissues, which makes it possible to take out the whole assembly: ablated vein VS olive+ traction probe 3 through the olive inlet incision.

Alternatively, the surgeon can extract the ablated vein by itself, through the incision made at the ankle, the traction probe and the olive being then led back and out through the incision made at the groin crease line.

The advantageous embodiment shown in FIGS. 10 through 14 differs from the one just described, first in that the longitudinal slot 9′ does not extend over the whole cylindrical anterior part 6 of the olive 1, but only from the ogival posterior portion 6a of the anterior part 6 of said olive up to the ogival rear end 7b of the posterior part 7 thereof.

In this case, the cylindrical skirt 12 of the anterior part 6 of the olive 1 is provided with a port 14, for example of circular shape, ending in the axial cavity 11 and communicating with the longitudinal slot 9′.

Based on this advantageous embodiment, the free anterior edge 12a of the cylindrical skirt 12 is continuous and does have any weak area; therefore, the circular pulling lip 12a is homogeneous over its circumference and so is the vein pulling action from the surrounding tissues.

According to this embodiment, one end of the traction probe 3 provided with an end fitting 5 can be inserted into the axial cavity 11, then taken out through the lateral port 14, then led into the longitudinal slot 9′, so as allow for said end fitting to enter into the axial compartment 10 of the olive, by sliding into said compartment from the open posterior end of the latter.

In addition, according to another embodiment, the circular front end of the posterior part 7 of the olive 1, for example consisting of a cylindrical portion 7a of said posterior part 7, is without the circular pulling lip 13 and without the cavity or annular pocket 8a.

1. Surgical instrument for ablation of diseased veins, particularly saphenous veins, said surgical instrument comprising.
a pulling component or pulling olive; the olive comprising:
an anterior part having a front end and being delimited by an annular groove, an axial cavity open at said front end of said anterior part;
a circular pulling lip delimited by said axial cavity; and
a posterior part with a circular section, being delimited by said annular groove, said circular section having a front circular end with a diameter greater than a diameter of said anterior part;
a traction probe being attached to the olive and having end fittings; and
a return thread in moving relation to the olive in opposite directions and located under said anterior part of the olive.

2. Surgical instrument according to claim 1, wherein said posterior part has a cylindro-ogival shape, the shape comprising a cylindrical front portion and an ogival rear portion.

3. Surgical instrument according to claim 2, wherein said anterior part has a rear portions connecting said anterior part said posterior part and having a roughly ogival shape.

4. Surgical instrument according to claim 2, wherein said cylindrical front portion is extended by a circular lip oriented, frontward, delimiting an annular pocket behind said annular groove.

5. Surgical instrument according to claim 3, the olive further comprising:
a lateral longitudinal slot extending over part of a length of the olive, said length including the ogival portion of said anterior part to said ogival rear portion, said slot communicating with an axial compartment open at ends thereof and being made in said posterior part said axial compartment being shaped to attach one end fitting of said traction probe, said slot communicating with a lateral port ending in said axial cavity.

6. Surgical instrument according to claim 1, the olive further comprising:
a lateral longitudinal slot extending over a whole length thereof, said slot communicating with an axial compartment open at ends thereof and being made in said posterior part said axial compartment being shaped to attach one end fittings of said traction probe.

7. Surgical instrument according to claim 1, wherein axial cavity has a depth at least equal to one fifth of a total length of the olive or equal to at least one third of a length of said anterior parts.

8. Surgical instrument according to claim 7, wherein said depth of the axial cavity is approximately equal to the length of the anterior part.

9. Surgical instrument according to claim 1, wherein said return thread is permanently integral with the pulling olive.
